(19) United States
(12) Patent Application Publication
(10) Pub. No.: US 2023/0407225 A1
(43) Pub. Date: Dec. 21, 2023

(54) METHODS AND SYSTEMS FOR PRODUCTION OF CELL CULTURE SCAFFOLDS

(71) Applicant: CORNING INCORPORATED, Corning, NY (US)

(72) Inventors: Ryan Thomas Adams, Charlotte, NC (US); Amy Marie Richter Blakeley, Warrenton, VA (US); Andrew William Doyle, East Waterboro, ME (US); Joseph William Mattson, Painted Post, NY (US)

(21) Appl. No.: 18/033,884

(22) PCT Filed: Oct. 26, 2021
(86) PCT No.: PCT/US2021/056608

§ 371 Date: Apr. 26, 2023

Related U.S. Application Data
(60) Provisional application No. 63/107,660, filed on Oct. 30, 2020.

(51) Int. Cl. C12M 1/12; C12N 5/00; C12M 3/06; C12M 3/00; C12M 1/00

(52) U.S. Cl. CPC C12M 25/16; C12N 5/0075; C12N 2537/10; C12M 23/44; C12M 29/04; C12M 23/16

(57) ABSTRACT

Systems and methods for the continuous production of digestible scaffolds for three-dimensional cell growth applications are provided. Systems for producing cell culture scaffolds include a housing and a plurality of modular components disposed in a serial arrangement within the housing, the modular components connected through a plurality of microfluidic flow channels. The modular components may comprise an emulsifier, a coating reactor, and a separator. Optionally, systems may comprise a porous membrane. Methods include cross-linking of polymer solutions into gels of discrete shapes; binding a layer of cell growth media to the gels; and drying or dehydrating the gels to form an aerogel functionalized for use as cell growth media.

### CROSS-REFERENCE TO RELATED APPLICATIONS

This application claims the benefit of priority under 35 U.S.C. § 119 of U.S. Provisional Application Serial No. 63/107,660 filed on Oct. 30, 2020, the content of which is relied upon and incorporated herein by reference in its entirety.

### TECHNICAL FIELD

The present specification generally relates to systems and methods of manufacture and, more specifically, to methods and systems of manufacturing scaffolds for use in cell culture.

### BACKGROUND

Cells cultured in three dimensions can exhibit more in vivo-like functionality than their counterparts cultured in two dimensions as monolayers. In 2D cell culture systems, cells are forced to adhere to a rigid surface and are geometrically constrained, adopting a flat morphology which alters the cytoskeleton regulation that is important in intracellular signaling, and consequently can affect cell growth, migration, and apoptosis. Moreover, organization of the ECM, which is significant to cell differentiation, proliferation, and gene expression, is absent in most 2D cells.

When cells are grown in 3D, the cells tend to interact with each other rather than attaching to the substrate. The additional dimensionality of 3D cultures is believed to lead to the differences in cellular responses because it influences the spatial organization of the cell surface receptors engaged in interactions with surrounding cells and induces physical constraints to cells. These spatial and physical aspects in 3D cultures are believed to affect the signal transduction from the outside to the inside of cells, and ultimately influence gene expression and cellular behavior. As such, cells cultured in 3D more closely resemble in vivo tissue in terms of cellular communication and the development of extracellular matrices.

To generate 3D cell cultures, the cells must be grown or cultured under appropriate conditions with cell culture media, which provides nutrients for cell growth. Some conventional 3D cell culturing methods involve using a bead or scaffold during culturing. However, existing production methods for such beads or scaffolds are time intensive and subject to significant bottlenecks within the process stages. Due to the type of equipment involved in processing, the existing production methods are slow, operator dependent, and difficult to change, resulting in variability within the manufacturing process.

### SUMMARY

Process speed and agility, coupled with consistent and repeatable manufacturing practices, are required for the successful production of scaffolds for use in cell culture. The systems and methods disclosed herein provide a repeatable, efficient process for generating coated microcarriers, which can be tuned to size and shape for a wide array of applications. Embodiments described herein remove variability introduced by manual operation of each significant process stage, reduce the risk of contamination by maintaining a closed system from gelation to drying, and maintain an aseptic environment by using alcohol solvents.

According to an aspect, a system for producing cell culture scaffolds comprises a housing; and a plurality of modular components disposed in a serial arrangement within the housing, the modular components connected through a plurality of microfluidic flow channels.

In some embodiments, the modular components may comprise an emulsifier, a coating reactor, and a separator.

In some embodiments, the emulsifier may comprise inputs comprising: an organic solution flow channel, and a crosslinking solution flow channel; and an output comprising an emulsification flow channel in communication with the coating reactor.

In some embodiments, the organic solution flow channel and the crosslinking solution flow channel are in communication through microfluidic pores disposed between the organic solution flow channel and the crosslinking solution flow channel.

In some embodiments, the emulsifier further comprises a porous membrane. In some embodiments, a critical dimension of each cell culture scaffold is determined by a pore size of the porous membrane. In some embodiments, the porous membrane is removable and interchangeable.

In some embodiments, the system further comprises an organic solution stock having an input line to the emulsifier; and a crosslinking solution stock having an input line to the emulsifier. In some embodiments, the system further comprises a pump disposed between the organic solution stock and the emulsifier. In some embodiments, the organic solution stock input line further comprises a mass flow controller.

In some embodiments, the system further comprises a pump disposed between the crosslinking solution stock and the emulsifier. In some embodiments, the crosslinking solution stock input line further comprises a mass flow controller. In some embodiments, a flow rate of the crosslinking solution is greater than or equal to a flow rate of the organic solution.

In some embodiments, the organic solution comprises a polymer solution or a sugar solution. In some embodiments, the organic solution comprises a polygalacturonic acid (PGA) solution.

In some embodiments, the organic solution comprises oils, nonpolar fluids, alcohols, water, surfactants, or any combination thereof. In some embodiments, the nonpolar fluids may comprise nonpolar hydrocarbons, other nonpolar fluids, or a combination thereof. In some embodiments, the alcohols may comprise different chain length alcohols, mixtures of alcohols, mixtures of alcohols and water, or a combination thereof.

In some embodiments, the crosslinking solution comprises an ionic salt solution. In some embodiments, the ionic salt solution comprises an ionic calcium salt solution. In some embodiments, ethanol is the solvent in the ionic calcium salt solution.

In some embodiments, the coating reactor comprises inputs comprising the emulsification flow channel in communication with the emulsifier, and a coating solution flow channel intersecting the emulsification flow channel; and an output comprising a coated emulsification flow channel in communication with the separator.

In some embodiments, the system further comprises a coating solution stock having an input line to the coating reactor. In some embodiments, the system further comprises a pump disposed between the coating solution stock and the coating reactor. In some embodiments, the coating solution stock input line further comprises a mass flow controller. In some embodiments, the coating solution comprises a polymer coating solution or a peptide coating solution. In some embodiments, the coating reactor is a continuous flow coating reactor.

In some embodiments, the inputs to the separator comprise the coated emulsification flow channel in communication with the coating reactor; and a supercritical fluid supply in communication with the coated emulsification flow channel.

In some embodiments, the outputs from the separator comprise a solvent evaporation channel, wherein solvents from the coated emulsification are evaporated and removed by the supercritical fluid; and solids comprising cell culture scaffolds.

In some embodiments, the supercritical fluid comprises supercritical CO₂. In some embodiments, the system further comprises a CO₂ stock and pressure regulator. In some embodiments, the CO₂ stock and pressure regulator are external to the housing.

In some embodiments, the system further comprises an alcohol stock and pressure regulator. In some embodiments, the alcohol stock and pressure regulator are external to the housing. In some embodiments, the alcohol in the alcohol stock comprises ethanol. In some embodiments, the alcohol from the alcohol stock is supplied to a first alcohol wash disposed between the emulsifier and the coating reactor, wherein an emulsification fluid is washed with alcohol after leaving the emulsifier and before entering the coating reactor.

In some embodiments, the alcohol from the alcohol stock is supplied to a second alcohol wash disposed between the coating reactor and the separator, wherein a coated emulsification fluid is washed with alcohol after leaving the coating reactor and before entering the separator.

In some embodiments, the cell culture scaffolds comprise animal-free, digestible cell culture media substrates.

In some embodiments, the cell culture scaffolds comprise a polymer bead or slug.

In some embodiments, the cell culture scaffolds comprise dissolvable microcarriers (DMCs). In some embodiments, the dissolvable microcarriers are dissolvable or digestible by an enzyme or chelating agent. In some embodiments, each DMC comprises a critical dimension of about 300 μm or less.

In some embodiments, the system is closed from the atmosphere outside of the housing and aseptic.

According to an aspect, a method of producing cell culture scaffolds comprises crosslinking an aqueous organic solution into shaped gels; binding a layer or coating of a cell growth media to the shaped gels; and drying the coated shaped gels to form cell culture scaffolds comprising aerogels functionalized for use as cell growth media.

In some embodiments, the aqueous organic solution comprises a polymer solution or a sugar solution. In some embodiments, the aqueous organic solution comprises a polygalacturonic acid (PGA) solution.

In some embodiments, the organic solution comprises oils, nonpolar fluids, alcohols, water, surfactants, or any combination thereof. In some embodiments, the nonpolar fluids may comprise nonpolar hydrocarbons, other nonpolar fluids, or a combination thereof. In some embodiments, the alcohols may comprise different chain length alcohols, mixtures of alcohols, mixtures of alcohols and water, or a combination thereof.

In some embodiments, the method comprises continuous production of cell culture scaffolds. In some embodiments, the cell culture scaffolds are for three-dimensional cell growth applications. In some embodiments, the cell culture scaffolds comprise digestible cell culture scaffolds.

In some embodiments, the crosslinking, binding, and drying steps are modular processes that occur within a single device.

In some embodiments, the crosslinking step comprises introducing the aqueous organic solution to a crosslinking solution through microfluidic channels or pores to form shaped gels in an emulsion, wherein the emulsion comprises the shaped gels as a dispersed phase and a solvent as a continuous phase.

In some embodiments, the crosslinking solution comprises a salt solution. In some embodiments, the salt solution comprises a calcium salt solution comprising CaCl₂, CaCO₃, CaSO₄, or a combination thereof in alcohol.

In some embodiments, the method further comprises exposing the shaped gels to an alcohol washing stage.

In some embodiments, the binding step comprises binding a cell growth medium to the shaped gel through a cross-linking reaction facilitated by a crosslinking reagent.

In some embodiments, the cell growth medium comprises a polymer coating medium or a peptide coating medium.

In some embodiments, the method further comprises exposing the coated shaped gels to an alcohol washing stage.

In some embodiments, the drying step further comprises using small pores or membranes in microchannels to separate the coated shaped gels of the dispersed phase from the solvent of the continuous phase of the emulsion.

In some embodiments, the coated shaped gels are carried to a separation vessel. In some embodiments, the solvent is removed from the coated shaped gels. In some embodiments, the solvent is removed from the coated shaped gels through supercritical fluid extraction. In some embodiments, the method further comprises depressurizing and reclaiming the solvent.

Additional features and advantages of the embodiments described herein will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments and are incorporated into and constitute a part of this specification. The drawings illustrate the various embodiments described herein, and together with the description, serve to explain the principles and operations of the claimed subject matter.

### BRIEF DESCRIPTION OF THE DRAWINGS

FIG. 1 shows a schematic of a system according to embodiments described herein.

FIG. 2 shows a schematic of a system according to embodiments described herein.

FIG. 3 shows a process flow diagram according to embodiments described herein.

FIG. 4 shows a schematic of a bead generation step according to embodiments described herein.

Reference will now be made in detail to embodiments of systems and methods of producing cell culture scaffolds, examples of which are illustrated in the accompanying drawings. Whenever possible, the same reference numerals will be used throughout the drawings to refer to the same or like parts.

### DETAILED DESCRIPTION

Embodiments described herein include the continuous production of digestible scaffolds for three-dimensional cell growth applications. Systems and methods according to embodiments described herein incorporate a serial, modular arrangement of microchannel formers, reactors, and separation vessels for the continuous production of coated aerogels, specifically for three-dimensional cell growth applications. More specifically, embodiments described herein provide systems and methods for cross-linking (also referred to as gelation herein) of polysaccharide solutions into gels of discrete shapes; binding a layer of cell growth media to the gels; and drying or dehydrating the gels to form an aerogel functionalized for use as cell growth media.

Conventional production methods for the cell culture scaffolds are divided into three overarching unit operations or batch process stages of gelation, coating, and drying. The production methods are time intensive and significant bottlenecks are described with reference to each process stage. For example, in conventional production methods, an alginate, a starch solution, or a polysaccharide solution, is administered dropwise into a bath of aqueous salt. The cations interact with individual monomers, dimers, trimers, etc., in an alginate, a starch solution, or a polysaccharide solution, causing them to crosslink and form a hydrogel. The hydrogel droplets retain their spherical shape during the crosslinking process, resulting in relatively monodispersed hydrogel beads. These beads are exposed to alcohol rinses to displace any water in the resulting hydrogel, forming an alcohol-based gel (“alcogel”). The alcogels are held in a batch reactor before being “coated” with a cell growth medium. Here, “coating” may be a true coating process and may also include phenomena where the cell growth media physically and/or chemically bond with the surface of the gel. In some examples, cell growth medium is added to the reactor to attach to the surface of the gels, which provides a coating layer to the gel that is specifically functionalized for cell growth. The coated beads may be subject to a number of alcohol washes downstream of the reaction. After the coating process, the wet, coated beads (alcogels) are subject to a series of drying steps, such as a heated, rotating evaporation step where wet alcogels are placed into a rotating evaporator and dried under significant heat and vacuum for several hours. The dry beads may then be sent to a freeze-drying step (lyophilization) to completely remove all water from the material, which can take upwards of several days.

In contrast to the batch production methods used in conventional techniques, the systems and methods described herein allow for on-demand, continuous production of scaffolds for three-dimensional cell growth applications. Systems and methods described herein may be used for rapid production of animal-free, digestible cell culture media substrates through a modular arrangement of emulsification technology, coating reactors, and separation vessels. In embodiments, the cell culture scaffolds may comprise digestible cell culture media substrates. In some embodiments, the cell culture scaffolds may be less than 300 microns (μm) in their critical dimension. The system incorporates microfluidic pores and flow channels to allow for the small length scale required by the culture media. In embodiments, the systems and methods may be used to produce dissolvable microcarrier (DMC) beads. In embodiments, the systems and methods may be used for the production of polymer beads or slugs, and may include in-situ formation, coating, and drying of such polymer beads or slugs.

In embodiments, methods described herein provide cross-linking (also referred to as gelation herein) of polysaccharide solutions into gels of discrete shapes; binding a layer of cell growth media to these gels; and drying or dehydrating these gels to form an aerogel functionalized for use as cell growth media.

Systems according to embodiments described herein provide efficient, effective production of cell culture scaffolds. In some embodiments, the cell culture scaffolds are dissolvable cell culture scaffolds. In some embodiments, the cell culture scaffolds are coated aerogel beads. In some embodiments, the cell culture scaffolds are coated polymer beads or slugs. In some embodiments, the cell culture scaffolds are dissolvable microcarrier (DMC) beads. In some embodiments, methods and systems provide reliable, continuous production of dissolvable microcarriers (DMCs). Systems described herein rapidly produce these types of cell culture scaffolds through a serial arrangement of microfluidic emulsifiers, coaters, and separation vessels. Methods and processes comprise a formation step, followed by a coating step, followed by a drying step. In particular, the formation step comprises bead and droplet formation, the coating step comprises bead coating, and the drying step comprises bead drying.

Microfluidic channels or pores intersect flow profiles between an aqueous organic solution and a crosslinking solution. Nonlimiting examples of an aqueous organic solution include a polymer solution or a sugar solution. In some embodiments, the crosslinking solution is a salt solution. Nonlimiting examples of a salt solution include an ionic salt solution or a calcium salt solution. In general, the flow rate of the salt solution is greater than or equal to the flow of the organic solution. The interfacial tension between the two solutions encourages breakup of the organic solutions into droplets, slugs, or other dispersed shapes, creating an emulsion. In most cases, a large disparity exists between the surface tension values of the continuous phase and the dispersed phase. The emulsion comprises the dispersed organic solution droplets in the salt solution. The calcium ions in the salt solution act as a cross-linking agent, preserving the shape of the dispersed phase in the emulsion. The rate of cross-linking is controlled by using blends of ionic calcium salts (e.g. CaCl₂, CaCO₃, CaSO, etc.).

The emulsion is carried downstream to a continuous-flow coating reactor, where a polymer or peptide solution either coats or binds to the surface of the dispersed phase, often creating a functionalized surface. The coating solution intersects the flow of the emulsion, forcing laminar or turbulent mixing between the solution. Typically, the coating solution will exhibit a relative philicity towards the dispersed phase and a relative phobicity toward the continuous phase. This affinity to the dispersed phase will encourage the coating solution to encapsulate or otherwise adhere to the dispersed phase. The addition of cross-linking agents will encourage a chemical bond between the coating solution and the dispersed phase, establishing a robust coating layer.

The emulsion, comprised of a continuous phase and a coated dispersed phase (the cross-linked organic solution having a coating solution coating or layer), flows toward a separation vessel designed to leverage supercritical fluid extraction (SFE) or other extraction technology. The solvent in the continuous phase is removed from the system by the chosen extraction technology. In some embodiments, the solvent is an alcohol. The solvent is removed from the continuous phase and from within the cross-linked matrix of the dispersed phase, resulting in a polymer-coated aerogel. In some embodiments, the extraction technology is SFE, due to its high yield in recycling the extracted solvent. In some embodiments, the consumption of the solvent in continuous phase is minimal.

Unlike conventional technologies, embodiments described herein do not rely on operator intervention. The system is closed from the atmosphere (e.g., the atmosphere outside of the housing), reducing the potential sources of contamination and also reducing the potential for external process perturbations. Moreover, an aseptic atmosphere is provided (e.g. the atmosphere within the housing), due to the presence of largely alcohol-based solutions. Furthermore, systems and processes according to embodiments described herein do not rely on long, time consuming or energy-intensive drying processes, but instead provide for real-time, in-situ monitoring and control of process variables. Embodiments described herein also allow for cost savings due to a reduced footprint and reduced process complexity, operating expense reduction due to reduced operator time, reduced waste from real-time monitoring of process variables and characteristics, and operating expense reduction due to improved use of expensive coatings. In addition, the systems and processes provide a high degree of control over bead size, as well as a high degree of control over the coating layer. The process is configured as on-demand and can conform immediately to user requests.

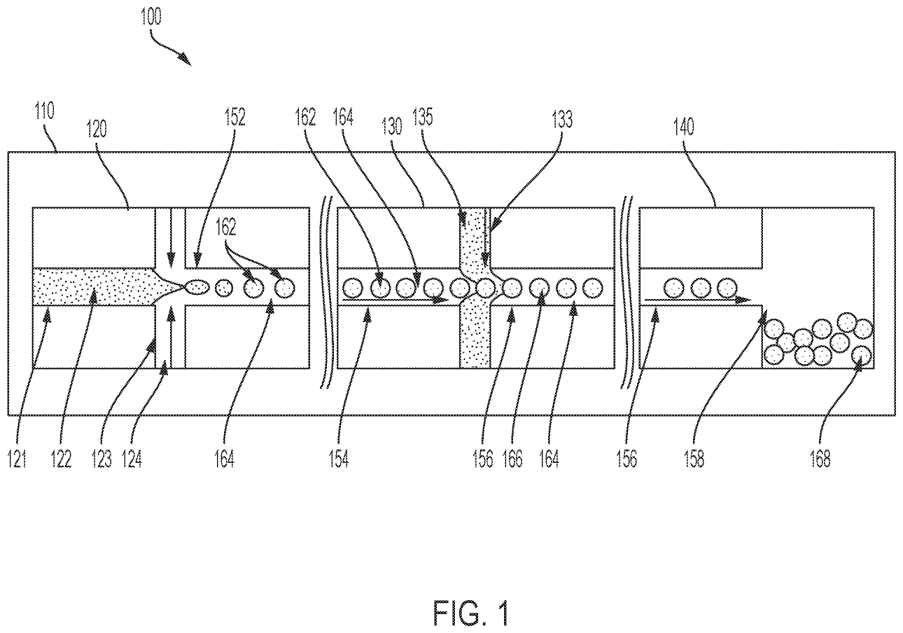

FIG. 1 shows a schematic of a system 100 according to an embodiment described herein. As shown in FIG. 1, an emulsifier 120, a coating reactor 130, and a separator 140 are disposed within a housing 110. Within the emulsifier 120, a fluid channel 121 for an organic solution 122 is shown with a fluid channel 123 for crosslinking solution 124 intersecting that of the organic solution 122. In this embodiment, the organic solution 122 is PGA in water and the crosslinking solution 124 is calcium in ethanol. At the intersection, the two solutions combine to form an emulsion in microfluidic channel 152. The interfacial tension between the two solutions encourages breakup of the PGA solution into droplets or PGA beads 162. The beads 162 are the dispersed phase (DP) of the emulsion in the continuous phase (CP) of the crosslinking solution 164. The emulsion is carried in an emulsification flow channel 154 downstream to the coating reactor 130. The coating reactor 130 comprises a coating solution flow channel 133 that intersects the emulsification flow channel 154. The coating solution 135 coats or binds to the surface of the PGA beads 162 to create a functionalized surface. In some embodiments, the coating solution 135 comprises a cell growth medium, such as Synthemax II® (Corning Incorporated, Corning, NY) (referred to as SMII herein) described in US 2019/0153256, the contents of which are incorporated by reference herein in their entirety. The emulsion now comprises a continuous phase 164 and a coated dispersed phase 166, which is carried in a coated emulsification flow channel 156 downstream to the separator 140. Within the separator 140, is a flow channel 158 comprising a supercritical fluid. In this embodiment, the supercritical fluid is CO₂. The supercritical CO₂ removes the solvent from the emulsion, thereby separating the ethanol and CO₂ from the coated PGA beads 168. The system 100 may further comprise bead storage (not shown) for storing the dried, coated beads after treatment in the separator 140. The system 100 may further comprise a controller (not shown) in communication with the system 100 for monitoring and control of flow rates, pressure, temperature, sensors, and any other system control features.

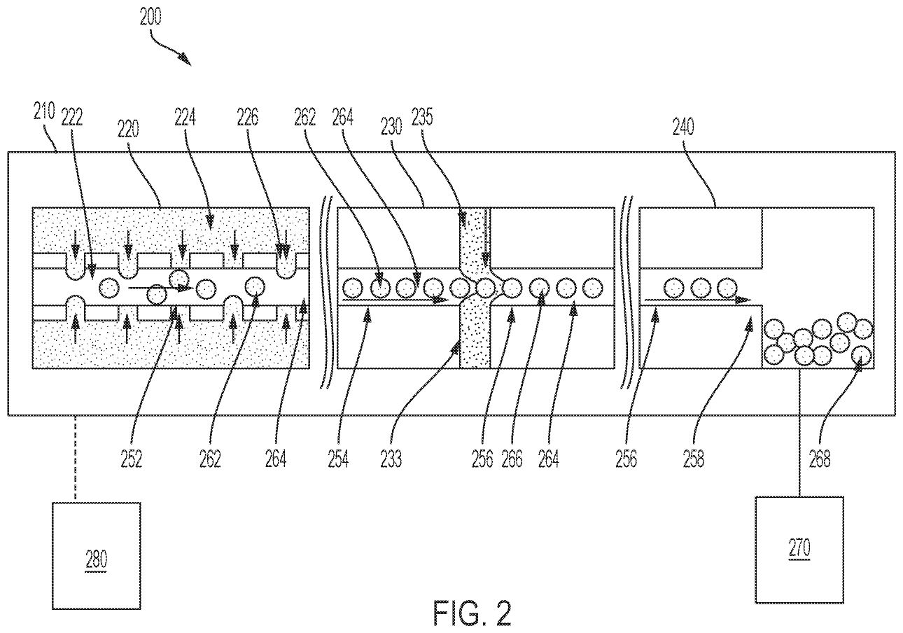

FIG. 2 shows a schematic of a system 200 according to an embodiment described herein. As shown in FIG. 2, an emulsifier 220, a coating reactor 230, and a separator 240 are disposed within a housing 210 and connected via microfluidic flow channels 252, 254, 256, 258. The system and method may be divided into three modular process units: gel forming in the emulsifier 220, gel coating in the coating reactor 230, and gel drying in the separator 240. Within the emulsifier 220, a crosslinking solution 222 is administered to a microfluidic channel 252. For example, the crosslinking solution 222 may comprise calcium in ethanol. An organic solution 224 is pumped into the same channel 252 at an angle not opposing the flow. For example, the organic solution 224 may comprise PGA in water. The PGA intersects the calcium solution through a small channel or pore 226, and surface tension and shear forces facilitate the breakup of the PGA solution into beads 262. The beads 262 are the dispersed phase (DP) of the emulsion in the continuous phase (CP) of the crosslinking solution 264. The emulsion is carried in an emulsification flow channel 254 downstream to the coating reactor 230. The coating reactor 230 comprises a coating solution flow channel 233 that intersects the emulsification flow channel 254. The coating solution 235 coats or binds to the surface of the PGA beads 262 to create a functionalized surface. For example, the coating solution may comprise a cell growth medium, such as Synthemax II® (Corning Incorporated, Corning, NY). The emulsion now comprises a continuous phase 264 and a coated dispersed phase 266, which is carried in a coated emulsification flow channel 256 downstream to the separator 240. Within the separator 240, is a flow channel 258 comprising a supercritical fluid. For example, the supercritical fluid may comprise CO₂. The supercritical CO₂ removes the solvent from the emulsion, thereby separating the ethanol and CO₂ from the coated PGA beads. The ethanol and CO₂ may then be output from the separator and optionally recycled, with the dried, coated PGA beads resulting. The system may further comprise storage 270 for the dried, coated PGA beads 268. In some embodiments, the bead storage may be internal to the housing (not shown). In some embodiments, the bead storage may be external to the housing. The system 200 further comprises a controller 280. The controller 280 is in communication with the system 200 for monitoring and control of flow rates, pressure, temperature, sensors, and any other system control features.

Systems according to embodiments described herein may comprise a controller. The controller may be used to monitor and control sensors, flow rates, pressure, temperature, and other controllable features within the system. In some embodiments, the controller may communicate wirelessly with components of the system. The controller may comprise memory and a processor. In some embodiments, the controller is in communication with a user interface.

The controller may process data acquired from the system components. The data may include pressure, temperature, and/or flow rate data. The controller may receive data through a processor from pumps or sensors or otherwise manipulate the data into parameters specified by a user. For example, a user may use a user interface to select various parameters or specifications for the modules. Data received and/or processed may be transferred to a display device for further processing and/or display. The data transfer may occur through a data interface, such as a data link or USB connection. The controller may also include a memory. The memory may be used to store data. The data may be unprocessed or processed data. Data stored may be downloaded to an external computer for processing and may be processed offline.

In some embodiments, the system may further comprise an imaging component, such as a camera or a scattering system for in-line monitoring of bead formation and particle size, and/or measurement of optical clarity.

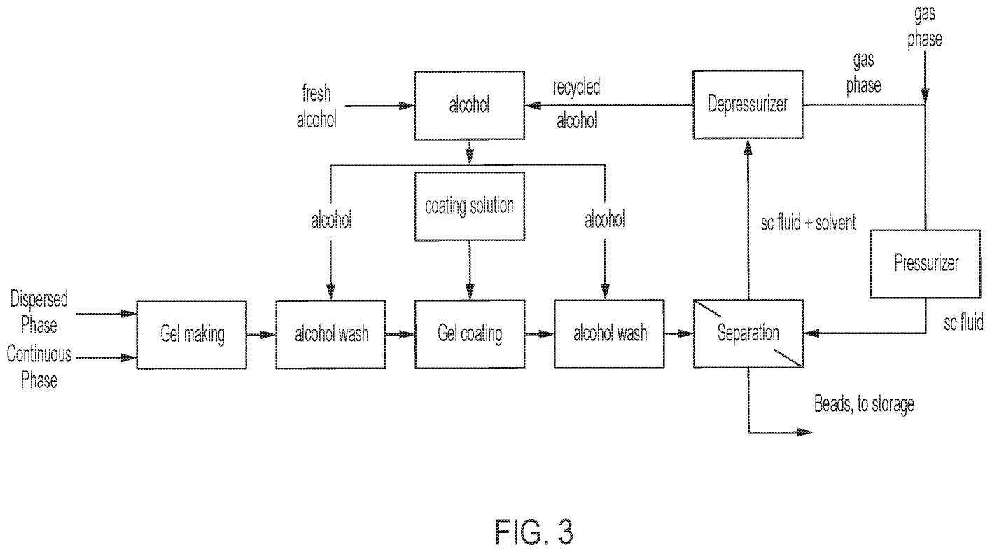

FIG. 3 shows a process flow diagram (PFD) according to embodiments described herein. In particular, the PFD for the continuous manufacture of coated aerogel beads is provided. The entire PFD may occur in a single unit, with the gel forming, washing, coating, and separation processes being modular processes. Pressure regulation and mass supplementation of gas (for example, CO₂) and alcohol (for example, ethanol) may be included in embodiments. In some embodiments, pressure regulation and mass supplementation of CO₂ and ethanol are external to this process.

In the gel forming process, an about 0.5-3 wt. % aqueous organic solution may be used as the dispersed phase. In some embodiments, the aqueous organic solution may comprise a 1.7 wt. % solution of polygalacturonic acid (PGA). In some embodiments, an about 3-6 wt. % crosslinking solution may be used as the continuous phase. In some embodiments, the crosslinking solution comprises a 4 wt. % solution of calcium salt (for example, a mixture of CaCl₂, CaCO₃, and/or CaSO₄) in alcohol (for example, ethanol) is used as the continuous phase. If the interfacial energy in the system is low, as it is in water-alcohol systems, droplets will not spontaneously form. In some embodiments, in order to form droplets, the stable jet formed from the dispersed phase may be perturbed (e.g. through a piezoelectric or other device). In some embodiments, the dispersed phase is mechanically perturbed (e.g., through vibrational or rotational forces). Some embodiments may use a PGA/glucose solution as the dispersed phase. The continuous phase will act as the cross-linking solution for the PGA solution. The calcium solution is administered via syringe pump (or other method) to a microfluidic channel at a rate between about 5 and about 200 mL/min. PGA solution is pumped into the same channel at an angle not opposing the flow at a rate of about 1 time to about 40 times less than the flow rate of the calcium solution. Flow rate through a single channel or nozzle may vary. The PGA intersects the calcium solution through a small channel or pore. Surface tension and shear forces facilitate the breakup of the PGA solution into beads, droplets, or slugs. In embodiments, the beads, droplets, or slugs may be about 100 to about 500 microns (μm) in their critical dimension.

The shape and size of the PGA in the dispersed phase is dependent on the channel size and flow rates of each solution. In some embodiments, PGA polymer beads are formed. In some embodiments, PGA “slugs” are formed. Here, the walls of the gel making phase are sufficiently small and the flow rate of the dispersed phase (DP) and continuous phase (CP) sufficient to produce oblong shapes, such as “bullet” or ellipsoidal shapes.

Surface tension is an important factor affecting bead formation, such as size, shape, and consistency of beads. In some embodiments, the organic solution may comprise any suitable fluid for the formation of beads or scaffolds. In some embodiments, the fluid may comprise oils, nonpolar hydrocarbons, other nonpolar fluids, different chain length alcohols, mixtures of alcohols, mixtures of alcohols and water, water, and surfactants may be used to control surface tension of the gelation media. In some embodiments, the bead may be of a spherical shape and the fluid may comprise oils, nonpolar hydrocarbons, and/or other nonpolar fluids. For example, addition of surfactant may allow achieving a round bead instead of an onion-shaped bead. Due to their influence on the mode of jet breakup, surfactants and surfactant concentrations may play a key role in the formation of microfluidic droplet. For example, increasing surfactant concentration may lend to the formation of long “threads” in the dispersed phase. Though the mechanism is not well understood, the presence of surfactant is accepted to influence the stability of liquid jets. Droplet formation may occur at the junction of the continuous and dispersed phases (i.e. droplet pinch-off), or it may occur due to breakup of the dispersed phase downstream of the junction (i.e. thread breakup), or through a number of other breakup modes. The degree to which surfactant is present in the system is assumed to influence the breakup mode.

The relative ratios of CaCl₂:CaCO₃:CaSO₄ determine the gelation kinetics of the PGA solution, and can be tuned to accommodate the specifications of the process and final product. Free calcium ions in solution facilitate the gelation of PGA and hold the shape of the dispersed phase after breakup. The gelled PGA/Ca²⁺/ethanol matrix forms an alcohol-based gel, or “alcogel”.

The alcogel is carried by the continuous phase to a coating microreactor. In some embodiments, the alcogel may be exposed to an alcohol washing stage to remove any calcium ions unconsumed during the gel making stage. In some embodiments, a polymer designed as an animal-free cell growth medium may be used a coating medium. In some embodiments, the coating medium may comprise Synthemax II® (SMII) (commercially available from Corning Incorporated, Corning, NY). In some embodiments, collagen may be used as a coating medium for alternative cell-growth applications. The coating medium is bound to the alcogel through a cross-linking reaction, which is generally facilitated by a crosslinking reagent, such as glutaraldehyde. The coating medium intersects the flow profile of the alcogel in solution. The flow rates of each species are dependent on the size of the channels in the coating microreactor and on the reaction kinetics of the coating reaction (or cross-linking, in this example). Systems and methods described herein may use solution rheology for tuning purposes. For example, the amount and grade of PGA in the solution may impact the beads formed. In embodiments, the width of the channel will generally be larger than the width of the alcogel, which eliminates shear on the polymer coating during the initial phases of cross linking when the polymer coat may be fragile. However, some embodiments may use wall shear to encourage rotation of the alcogel within the channel during the coating phase, which may yield a more uniform coating layer. The coating medium may be administered at a rate of 5-500 mL/min during the coating phase.

In some embodiments, the coated alcogels may be diverted to an alcohol wash to remove any unreacted SMII or other reagents in the coating process. Coated alcogels are carried by the continuous phase to the separation vessel to separate the gels from the continuous phase, and to remove any of the continuous phase from the gel matrix. Small pores or membranes in the microchannels separate the dispersed phase from the continuous phase. The dispersed phase is carried to the separation vessel, while the continuous phase is sent to reclamation or disposal. In this embodiment, the alcohol solvent is removed from the alcogel through supercritical fluid extraction (SFE) using supercritical CO₂ (sCO₂). However, any method of liquid/fluid extraction may be used to remove the alcohol from solution. In this embodiment, sCO₂ is formed from CO₂ gas at a pressure and temperature greater than 72.9 atm and 304.25 K, respectively. The coated alcogels are exposed to the sCO₂, which acts as a solvent for the alcohol in the alcogel matrix. The sCO₂ and alcohol mixture is carried to a depressurization vessel to form gaseous CO₂ and liquid alcohol. This alcohol can be reclaimed and reused for any of the solvation or washing stages. Dried, coated PGA aerogels are carried from the microfluidic system to a storage container or other holding unit.

In some embodiments, the sub-critical liquid CO₂ would be used to remove the solvent from solution. Alternatively, in some embodiments, the entire assembly of the system may be operated at temperatures and pressures above the critical point of CO₂, as operating at such temperatures and pressures may expedite the cross-linking reactions and reduce design complexity by making the entire assembly hospitable to supercritical CO₂ formation.

In some embodiments, CO₂ is the supercritical fluid used for ethanol/media extraction. In some embodiments, other supercritical fluids may be used for ethanol/other media extraction. Nonlimiting examples of other supercritical fluids include nitrogen and methane, which are gases that are supercritical at substantially lower pressures than CO₂, which may provide repressurization cost savings and a more mechanically stable environment for beads (i.e. lower pressure may lead to less mechanical deformation; potentially less impact on morphology of coated layers). The low temperatures required for maintaining N₂ or CH₄ as supercritical fluids could be “heat integrated” with the freeze-drying step thereby offsetting costs associated with maintaining a low temperature supercritical fluid.

In some embodiments, an ethanol removal/recovery method such as traditional thermally driven evaporation with a condenser may be used to recover ethanol. For example, such an ethanol removal and recovery method may be used to avoid undesirable effects on the beads from high pressure CO₂ (such as irreversible deformation of beads, undesirable changes in coating morphology, or delamination of coatings). In some embodiments, methods may include a treatment of the recovered alcohol or supercritical fluid to maintain the required level of purity.

In addition, in embodiments, uniform size distribution of the microcarriers may be provided. Uniform size distribution may ensure faster and cleaner separation of microcarriers from supernatant during use, which may make medium exchange and final production isolation more predictable, more reliable, and less expensive. In embodiments, microcarrier size can be precisely tuned to different ranges. This allows the settling speed of the beads to be customized to match different bioprocess needs without changing the material properties of the beads.

The microcarrier beads may be spherical or substantially spherical. The beads may be any suitable size, and systems and methods described herein are capable of tuning parameters to achieve different bead sizes. In some examples, a custom distribution of beads sizes may be achieved by said tuning for a targeted application. In some embodiments, the beads may have an average diameter ranging from 10 to 500 micrometers, e.g., 10, 20, 25, 50, 75, 100, 150, 200, 250, 300, 350, 400, 450, or 500 micrometers, including ranges between any of the foregoing values. In embodiments, microcarrier beads can be manufactured within narrow and specific size ranges. That is, they can be size-controlled. Control of the size of microcarrier beads is important for several reasons. If there is a wide size distribution, ranging from small to large microcarriers, microcarriers with smaller size will be in suspension much longer than larger size microcarriers. Exact settling time in the process would be much longer (because of the presence of smaller beads) or difficult to define. In use, more time will be required to ensure that the supernatant is clear from microcarriers.

Narrow size distribution enables settling of beads at a consistent speed which allows for more predictable separation of microcarriers from supernatant during medium exchange or culture produce isolation. Size of microcarriers can be fine-tuned to different ranges to control the settling speed. This enables customization of settling speed to match different process needs. Size controlled microcarriers have uniform surface area, which provides the same area available for cells to seed per microcarrier. This makes calculating the surface area available for cell seeding easier. In addition, cells will reach confluence at the same, or at a similar, time. As used herein, the terms “confluence” or “confluent” are used to indicate when cells have formed a coherent layer on a growth surface where all cells are in contact with other cells, so that virtually all the available growth surface is used. For example, “confluent” has been defined conventionally as the situation where all cells are in contact all around their periphery with other cells and no available substrate is left uncovered. The amount of a growth surface that is covered by cells may be referred to as a proportion of confluence. For example, a situation where approximately half of the growth surface is covered by cells is referred to herein as 50% confluence, or, in the alternative, as half confluence. Size-controlled microcarriers can be suspended in the same agitation conditions, which allows for fine control of shear force to balance good suspension of microcarriers and may allow conditions that cause less damage to cells. Well-defined settling times for different groups of size-controlled microcarriers can help easy separation during continuous cell culture to prevent uneven cell growth on beads fed at different times. For example, cells can be seeded on size-controlled microcarriers with 250 μm size first. After cells have reached half confluence, size-controlled microcarriers with of 350 μm size can be added in the bioreactor for bead-to-bead transfer. At the time of confluence for 250 μm microcarriers, microcarriers with this size can be removed by their unique settle speed or by filtration. Only beads with 350 μm size and half confluent are left in the bioreactor. Then, fresh 250 μm microcarriers can be added. After 350 μm microcarriers reach confluence, they can be collected and fresh 350 μm microcarriers added. This process may ensure that all the beads are removed when they reach confluence. In contrast, where microcarriers of the same size are used to do bead-to-bead transfer and continuous cell culture, cells on the beads from an earlier feeding will stay in bioreactor much longer than those on beads from a later feeding and the quality of cells can be deteriorated as a result of over confluence.

In embodiments, dissolvable microcarriers may be size-controlled during manufacture using a vibration encapsulator. Size-controlled beads may be formed by going through a microfluidic channel, nozzle, membrane, or mesh with a defined hole size, flow rate, and/or vibration frequency. The size of obtained beads may be controlled to a narrow range with a coefficient of variation of less than 10%.

Sizes of beads provided herein may be measured according to a hydrated measurement. As a nonlimiting example, a water-hydrated base measurement is used, such as measuring the bead after formation of the bead and before coating and drying.

According to systems and methods described herein, the emulsifier may comprise a microfluidic device for bead formation. The microfluidic device may be a microfluidic channel, a membrane, or a mesh. In some embodiments, bead size may be controlled with use of a mesh. A nonlimiting example of a mesh comprises a PET mesh with defined openings from about 25 microns to about 1000 microns in opening size. In some embodiments, the bead size may be further controlled with use of a membrane.

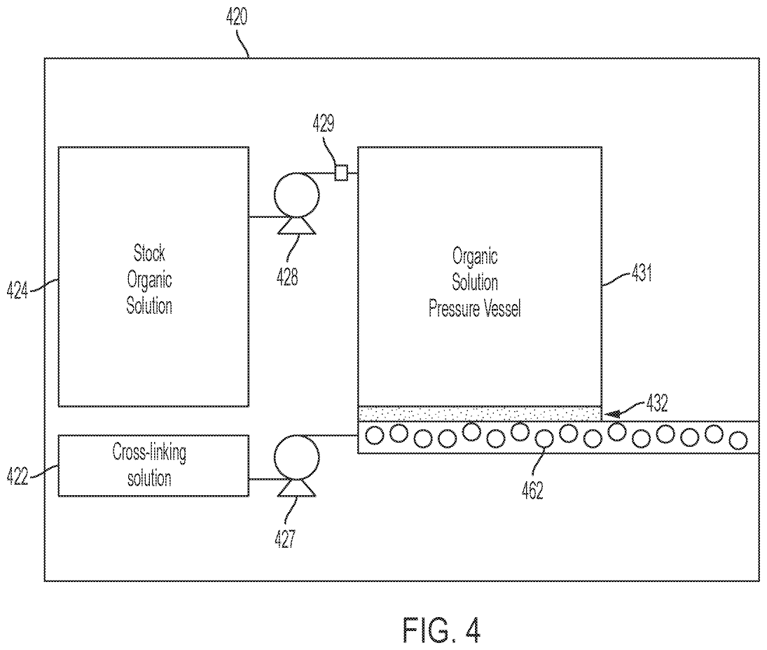

FIG. 4 shows a flow diagram for bead generation in an emulsifier 420 according to an embodiment. A crosslinking solution 422 (for example, calcium in an alcohol, such as ethanol) serves as the continuous phase and travels through pump 427 to a microfluidic flow channel. Organic solution stock 424 (for example, PGA stock solution) serves as the dispersed phase. The organic solution 424 travels through pump 428 and is controlled by a mass flow controller 429 while being pumped to an organic solution pressure vessel 431. The pressurized PGA solution flows through membrane 432 having a specified pore size to generate beads 462 of a known diameter in the microfluidic flow channel. The beads then flow downstream for further processing and coating.

Nonlimiting examples of membranes include membranes having a pore size of 0.2-0.8 microns, such as those manufactured by Membraflow, Germany; membranes having a pore size of 0.2-10 microns, such as those manufactured by Fairey Industrial Ceramics LTD, UK; membranes having a pore size of 0.05-14 microns, such as those manufactured by Asahi Glass Company, Japan; membranes having a pore size of 0.05-14 microns, such as those manufactured by Membralox, SCT, France; and membranes having a pore size of 7-60 microns, such as those manufactured by Micropore Technologies, LTD, UK.

In embodiments, the membrane may be removable and replaceable in order to tune pore size by substituting a membrane having a different pore size. Adjustments to mass flow of the crosslinking solution, PGA solution, or a combination thereof may be used to customize and control the final bead diameter.

Some embodiments of the present disclosure relate to methods of making dissolvable scaffolds for cell culture. In some embodiments, scaffolds as disclosed herein are described as being dissolvable and insoluble. As used herein, the term “insoluble” is used to refer to a material or combination of materials that is not soluble, and that remains crosslinked, under conventional cell culture conditions which include, for example, cell culture media. Also as used herein, the term “dissolvable” is used to refer to a material or combination of materials that is digested when exposed to an appropriate concentration of an enzyme that digests or breakdowns the material or combination of materials. Dissolvable scaffolds as described herein are porous scaffolds having an open pore architecture and highly interconnected pores. The pores of the scaffolds provide a protected environment for the culturing of cells where the cell-to-cell interactions and formation of ECM in a 3D fashion are aided. The dissolvable scaffolds may be completely digested which allows for harvesting cells without damaging the cells using protease treatment and/or mechanical harvesting techniques.

In embodiments, the systems and methods may be used to produce digestible or dissolvable microcarrier (DMC) beads. In some embodiments, the scaffolds comprise dissolvable microcarriers (DMCs) for use as a cell growth media. In some embodiments, the microcarriers comprise DMCs coated with Synthemax® II-SC (commercially available from Corning, Incorporated, Corning, NY), such as those described in International Publication Numbers WO2016/200888 and WO 2019/104069, the contents of each of which are incorporated by reference herein in their entirety. Digestible cell culture articles are disclosed in International Publication Number WO2014/209865, the content of which is incorporated herein by reference in its entirety.

In embodiments, cell culture articles formed from the systems and methods described herein may promote cell attachment and growth. PGA beads, due to their hydro gel nature and negative charge, do not readily support cell attachment without specific treatment. In order to promote attachment of anchorage dependent cells, the beads can be provided with a coating or other surface treatment. By way of example, the PGA beads can be functionalized with moieties promoting cell adhesion, for example, peptides such as those comprising a RGD sequence.

Further candidate peptides include those containing amino acid sequences potentially recognized by proteins from the integrin family, or leading to an interaction with cellular molecules able to sustain cell adhesion. Examples include BSP, vitronectin, fibronectin, laminin, Type I and IV collagen, denatured collagen (gelatin), and like peptides, and mixtures thereof. Further example peptides are BSP and vitronectin (VN) peptides.

In embodiments, the beads are surface functionalized with cell adhesion promoting recombinant proteins, which can be grafted or applied as a coating. Example recombinant proteins include fibronectin-like engineered proteins marketed under the trade names ProNectin® and ProNectin® plus, though other recombinant proteins that promote attachment of anchorage dependent cells can be used.

According to embodiments of the present disclosure, scaffolds as described herein may further include an adhesion polymer coating. The adhesion polymer may include peptides. Exemplary peptides may include, but are not limited to BSP, vitronectin, fibronectin, laminin, Type I and IV collagen, denatured collagen (gelatin), and like peptides, and mixtures thereof. Additionally, the peptides may be those having an RGD sequence. The coating may be, for example, Synthemax® II-SC (commercially available from Corning, Incorporated, Corning, NY).

In some embodiments, the organic solution may comprise a polysaccharide solution. In some embodiments, the polysaccharide solution may comprise a polygalacturonic acid (PGA) solution. Generally, polysaccharides possess attributes beneficial to cell culture applications. Polysaccharides are hydrophillic, non-cytotoxic and stable in culture medium. Examples include pectic acid, also known as polygalacturonic acid (PGA), or salts thereof, partly esterified pectic acid or salts thereof, or partly amidated pectic acid or salts thereof. Pectic acid can be formed via hydrolysis of certain pectin esters. Pectins are cell wall polysaccharides and in nature have a structural role in plants. Major sources of pectin include citrus peel (e.g., peels from lemons and limes) and apple peel. Pectins are predominantly linear polymers based on a 1,4-linked alpha-D-galacturonate backbone, interrupted randomly by 1,2-linked L-rhamnose. The average molecular weight ranges from about 50,000 to about 200,000 Daltons.

In some embodiments, the PGA solution may comprise a polygalacturonic acid chain of pectin that is partly esterified, e.g., methyl groups and the free acid groups may be partly or fully neutralized with monovalent ions such as sodium, potassium, or ammonium ions. Polygalacturonic acids partly esterified with methanol are called pectinic acids, and salts thereof are called pectinates. The degree of methylation (DM) for high methoxyl (HM) pectins can be, for example, from 60 to 75 mol % and those for low methoxyl (LM) pectins can be from 1 to 40 mol %. The degree of esterification of partly esterified polygalacturonic acids as described herein may be less than about 70 mol %, or less than about 60 mol %, or less than 50 mol %, or even less than about 40 mol %, and all values therebetween. Without wishing to be bound by any particular theory, it is believed that a minimum amount of free carboxylic acid groups (not esterified) facilitates a degree of ionotropic crosslinking which allow for the formation of a dissolvable scaffold which is insoluble.

In some embodiments, the PGA solution may comprise a polygalacturonic acid chain of pectin that is partly amidated. Polygalacturonic acids partly amidated may be produced, for example, by treatment with ammonia. Amidated pectin contains carboxyl groups (˜COOH), methyl ester groups (˜COOCH₃), and amidated groups (—CONH₂). The degree of amidation may vary and may be, for example, from about 10% to about 40% amidated.

According to some embodiments, dissolvable scaffolds may include a mixture of pectic acid and partly esterified pectic acid. Blends with compatible polymers may also be used. For example, pectic acid and/or partly esterified pectic acid may be mixed with other polysaccharides such as dextran, substituted cellulose derivatives, alginic acid, starches, glycogen, arabinoxylans, agarose, etc. Glycosaminoglycans like hyaluronic acid and chondroitin sulfate, or various proteins such as elastin, fibrin, silk fibroin, collagen and their derivatives can be also used. Water soluble synthetic polymers can be also blended with pectic acid and/or partly esterified pectic acid. Exemplary water soluble synthetic polymers include, but are not limited to, polyalkylene glycol, poly(hydroxyalkyl(meth)acrylates), poly(meth)acrylamide and derivatives, poly(N-vinyl-2-pyrrolidone), and polyvinyl alcohol.

Dissolvable scaffolds as described herein may be crosslinked to increase their mechanical strength and to prevent the dissolution of the scaffolds when placed in contact with cell culture medium. Crosslinking may be performed by ionotropic gelation as described below wherein ionotropic gelation is based on the ability of polyelectrolytes to crosslink in the presence of multivalent counter ions to form crosslinked scaffolds. Without wishing to be bound by any particular theory, it is believed that ionotropic gelation of the polysaccharide of the dissolvable scaffolds is the result of strong interactions between divalent cations and the polysaccharide.

In some embodiments, the dissolvable scaffold is a dissolvable foam scaffold. For example, a dissolvable foam scaffold may comprise a porous foam that includes an open pore architecture. Dissolvable porous foam scaffolds as described herein may have a porosity of from about 85% to about 96%. For example, foam scaffolds as described herein may have a porosity of from about 91% to about 95%, or about 94% to about 96%. As used herein, the term “porosity” refers to the measure of open pore volume in the dissolvable scaffold and is referred to in terms of % porosity, wherein % porosity is the percent of voids in the total volume of the dissolvable foam scaffold. Foam scaffolds as described herein may have an average pore size diameter of between about 50 μm and about 500 μm. For example, average pore size diameter may be between about 75 μm and about 450 μm, or between about 100 μm and about 400 μm, or even between 150 μm and about 350 μm and all values therebetween. Dissolvable scaffolds provide a protected environment within the pores of the scaffold for the culturing of cells. Additionally, dissolvable scaffolds may be dissolved when exposed to an appropriate enzyme that digests or breakdowns the material which facilitates harvesting of the cells cultured in the scaffold without damaging the cells. Scaffolds as described herein may have a wet density of less than about 0.40 g/cc. For example, scaffolds as described herein may have a wet density of less than about 0.35 g/cc, or less than about 0.30 g/cc, or less than about 0.25 g/cc. Scaffolds as described herein may have a wet density of between about 0.16 g/cc and about 0.40 g/cc, or between about 0.16 g/cc and about 0.35 g/cc, or between about 0.16 g/cc and about 0.30 g/cc, or even between about 0.16 g/cc and about 0.25 g/cc, and all values therebetween. Scaffolds as described herein may have a dry density of less than about 0.20 g/cc. For example, scaffolds as described herein may have a dry density of less than about 0.15 g/cc, or less than about 0.10 g/cc, or less than about 0.05 g/cc. Scaffolds as described herein may have a dry density of between about 0.02 g/cc and about 0.20 g/cc, or between about 0.02 g/cc and about 0.15 g/cc, or between about 0.02 g/cc and about 0.10 g/cc, or even between about 0.02 g/cc and about 0.05 g/cc, and all values therebetween.

Several pore types are possible in scaffolds. Open pores allow for cellular access on both sides of the scaffold and allow for liquid flow and transport of nutrients through the dissolvable scaffold. Partially open pores allow for cellular access on one side of the scaffold, but mass transport of nutrients and waste products is limited to diffusion. Closed pores have no openings and are not accessible by cells or by mass transport of nutrients and waste products. In some embodiments, the cell culture scaffolds comprise dissolvable foam scaffolds having an open pore architecture and highly interconnected pores. Generally, the open pore architecture and highly interconnected pores enable migration of cells into the pores of the dissolvable scaffolds and facilitate enhanced mass transport of nutrients, oxygen, and waste products. The open pore architecture also influences cell adhesion and cell migration by providing a high surface area for cell-to-cell interactions and space for ECM regeneration.

Cell culture articles produced from the systems and methods described herein may further allow for cell harvesting without the use of protease. Example cell culture articles are microcarriers, which are also referred to as beads or microbeads (collectively “microcarriers”). In embodiments, the cell culture article is a smooth and transparent (or translucent) bead comprising a gel that includes pectic acid, partially esterified pectic acid, or salts thereof. The cell culture articles may be spherical or substantially spherical and are formed by gelation. The calcium content of the cell culture articles may be adjusted to afford rapid cell harvesting under mild conditions that mitigates damage to the cells. Molecules promoting the attachment of anchorage-dependent cells may be attached to the surface of the cell culture article by chemical coupling or physical adsorption.

Non-proteolytic enzymes suitable for digesting the microcarrier, harvesting cells, or both, include pectinolytic enzymes or pectinases, which are a heterogeneous group of related enzymes that hydrolyze the pectic substances.

Cell harvesting involves contacting cell-laden microcarriers with a solution comprising a mixture of pectinolytic enzyme or pectinase and a divalent cation chelating agent. An example method for harvesting cultured cells comprises culturing cells on the surface of a microcarrier as disclosed herein, and contacting the cultured cells with a mixture of pectinase and a chelator to separate the cells from the microcarrier. Dissolvable scaffolds as described herein are digested when exposed to an appropriate enzyme, chelating agent, or combination thereof that digests or breakdowns the material. Non-proteolytic enzymes suitable for digesting the scaffolds, harvesting cells, or both, include pectinolytic enzymes or pectinases, which are a heterogeneous group of related enzymes that hydrolyze the pectic substances.

Pectinases (polygalacturonase) are enzymes that break down complex pectin molecules to shorter molecules of galacturonic acid. Pectinases catalyze the liberation of pectic oligosaccharides (POS) from polygalacturonic acid. Pectinases are produced by fungi, yeast, bacteria, protozoa, insects, nematodes and plants. Commercially-available sources of pectinases are generally multi-enzymatic, such as Novozyme Pectinex™ ULTRA SPL, a pectolytic enzyme preparation produced from a selected strain of Aspergillus aculeatus. Novozyme Pectinex™ ULTRA SPL contains mainly polygalacturonase, (EC 3.2.1.15) pectintranseliminase (EC 4.2.2.2) and pectinesterase (EC: 3.1.1.11). The EC designation is the Enzyme Commission classification scheme for enzymes based on the chemical reactions the enzymes catalyze. Pectinases are known to hydrolyze pectin. They may attack methyl-esterified pectin or de-esterified pectin. The concentration of pectinolytic enzyme in the digestion solution may be 1 to 200 U, e.g., 1,2, 5, 10, 20, 50, 100, 150, or 200 U, including ranges between any of the foregoing.

According to embodiments of the present disclosure, digestion of the dissolvable scaffolds may also include exposing the scaffold to a divalent cation chelating agent. Exemplary chelating agents include, but are not limited to, ethylenediaminetetraacetic acid (EDTA), cyclohexanediaminetetraacetic (CDTA), ethylene glycol tetraacetic acid (ETGA), citric acid and tartaric acid. The chelating agent concentration in the digestion solution may be 1 to 200 mM, e.g., 20, 50, 100, 150, or 200 mM. To prevent cytotoxic side effects, the concentration of chelating agent in the digestion solution may be 10 mM or less, e.g., 1, 2, 5, or 10 mM, including ranges between any of the foregoing.

In embodiments, the total volume of the digestion solution comprising the pectinolytic enzyme and the chelating agent is less than 10 times the microcarrier volume, e.g., 1, 2, 4, 5 or 10 times the volume of the microcarriers including ranges between any of the foregoing values.

The time to complete digestion of dissolvable scaffolds as described herein may be less than about 1 hour. For example, the time to complete digestion of scaffolds may be less than about 45 minutes, or less than about 30 minutes, or less than about 15 minutes, or between about 1 minute and about 25 minutes, or between about 3 minutes and about 20 minutes, or even between about 5 minutes and about 15 minutes.

Depending of the digestion time, temperature, and amount of pectinolytic enzyme added, the extent of digestion beads can be selected or predetermined. It has been observed that cells detach from the microcarrier surface before the bead is fully digested. It is therefore possible to harvest cells with or without complete digestion of the beads. In embodiments where cells are harvested from partially-digested microcarriers, separation of the cells from remnant microcarriers may be done by one or more of filtration, decantation, centrifugation, and like processing.

Beads are readily digested when their calcium content is less than 2 g/l of moist beads, e.g., less than 2, 1.5, 1, 0.8 or 0.5 g/l. When the calcium content of the beads at the harvest stage is greater than 1 g/l, a greater volume and/or concentration of pectinolytic enzyme and divalent cation chelating agent can be used. The time for complete digestion may be less than one hour, e.g., 10, 15, 30 or 45 min. As used herein, the term “complete digestion” refers to digestion of microcarriers that results in a microcarrier particle count that complies with the particle count test as described in The United States Pharmacopeia and The National Formulary Section 788 (USP<788>) entitled “Particulate Matter in Injections”. As indicated in USP<788> a preparation complies with the test if the average number of particles present in the units tested does not exceed particles per mL equal to or greater than 10 μm and does not exceed 3 particles per mL equal to or greater than 25 um. In embodiments, the microcarrier particle count for particles having a size of greater than or equal to 10 um after digestion of the microcarriers is less than 10 particles, e.g., 0, 1, 2, 3, 4, 5, 6, 7, 8 or 9, including ranges between any of the foregoing. In embodiments, the microcarrier particle count for particles having a size of greater than or equal to 25 um after digestion of the microcarriers is less than 1 particle, e.g., 0, 0.1, 0.2, 0.3, 0.4, 0.5, 0.6, 0.7, 0.8 or including ranges between any of the foregoing.

As defined herein the “moist bead” volume is the volume of the bed of beads after decantation or centrifugation. The bed comprises swollen beads as well as interstitial water (i.e., water present between the swollen beads). According to measurements, moist beads contain 70 vol. % swollen beads and 30 vol. % interstitial water. The swollen beads contain 99% water for a 1% PGA solution, 98% water for a 2% PGA solution, 97% water for a 3% PGA solution, etc.

### EXAMPLE

In an embodiment, aqueous PGA at a range of about 0.5-3 wt. % (dispersed phase, DP) is cross-linked with a calcium-in-ethanol solution, made up from a salt mixture of 0-5% CaCO₃, CaSO₄, and 90-100% CaCl₂ dissolved into pure ethanol to about 1 g to about 10 g salt/100 g solution (continuous phase, CP). The CP flows through a 1/16″ diameter channel at a rate of 5-200 mL/min. The DP is flowed through a series of pores in the channel, tangent to or not opposing the flow rate of the CP—as in a cross-flow membrane emulsification configuration—at a total flow rate of 5-200 mL/min. Flow rate through a single channel or nozzle may vary. Perturbations may be applied to the DP to encourage uniform breakup if necessary (e.g., rotational or vibrational), and the perturbations are nominally of the frequencies predicted by Rayleigh. Gelation occurs after the PGA droplets, with a diameter of 100-500 microns and nearly spherical in shape, leave the pore but before the droplets have a chance to coalesce in the CP. The ratio of CaCO₃:CaCl₂:CaSO₄ is optimized for gelation time and gel optical clarity.

The solution is washed with ethanol at a rate equivalent to displace the ethanol in the bulk solution. The washed solution is sent to a coating reactor to coat each bead with SMII for a total surface area coverage of 60-100%. Reactor widths are large enough to prevent wall shear on the beads.

Coated beads are sent downstream to a washing stage, which parallels the first. These washed beads are first mechanically separated from the CP by membrane separation before being sent to the separation vessel where the beads are contacted with sCO₂ for a residence time of nominally 3s. The sCO₂ mass rates are on the order of 1 kg/min. The sCO₂ is depressurized to separate gaseous CO₂ from liquid ethanol, and the gaseous CO₂ is repressurized to supercritical CO₂ and liquid ethanol is reclaimed and used for the washing stages. Aerogel beads are slowly depressurized and sent to storage.

Illustrative Implementations

The following is a description of various aspects of implementations of the disclosed subject matter. Each aspect may include one or more of the various features, characteristics, or advantages of the disclosed subject matter. The implementations are intended to illustrate a few aspects of the disclosed subject matter and should not be considered a comprehensive or exhaustive description of all possible implementations.

Aspect 1 pertains to a system for producing cell culture scaffolds comprising: a housing; and a plurality of modular components disposed in a serial arrangement within the housing, the modular components connected through a plurality of microfluidic flow channels.

Aspect 2 pertains to the system of Aspect 1, wherein the modular components comprise an emulsifier, a coating reactor, and a separator.

Aspect 3 pertains to the system of Aspect 2, wherein the emulsifier comprises: inputs comprising: an organic solution flow channel, and a crosslinking solution flow channel; and an output comprising an emulsification flow channel in communication with the coating reactor.

Aspect 4 pertains to the system of Aspect 3, wherein the organic solution flow channel and the crosslinking solution flow channel are in communication through microfluidic pores disposed between the organic solution flow channel and the crosslinking solution flow channel.

Aspect 5 pertains to the system of Aspect 3, wherein the emulsifier further comprises a porous membrane.

Aspect 6 pertains to the system of Aspect 5, wherein a critical dimension of each cell culture scaffold is determined by a pore size of the porous membrane.

Aspect 7 pertains to the system of Aspect 5, wherein the porous membrane is removable and interchangeable.

Aspect 8 pertains to the system of Aspect 3, wherein the system further comprises: an organic solution stock having an input line to the emulsifier; and a crosslinking solution stock having an input line to the emulsifier.

Aspect 9 pertains to the system of Aspect 8, further comprising a pump disposed between the organic solution stock and the emulsifier.

Aspect 10 pertains to the system of Aspect 9, wherein the organic solution stock input line further comprises a mass flow controller.

Aspect 11 pertains to the system of Aspect 8, further comprising a pump disposed between the crosslinking solution stock and the emulsifier.

Aspect 12 pertains to the system of Aspect 11, wherein the crosslinking solution stock input line further comprises a mass flow controller.

Aspect 13 pertains to the system of Aspect 8, wherein a flow rate of the crosslinking solution is greater than or equal to a flow rate of the organic solution.

Aspect 14 pertains to the system of Aspect 3, wherein the organic solution comprises a polymer solution or a sugar solution.

Aspect 15 pertains to the system of Aspect 3, wherein the organic solution comprises a polygalacturonic acid (PGA) solution.

Aspect 16 pertains to the system of Aspect 3, wherein the organic solution comprises oils, nonpolar fluids, alcohols, water, surfactants, or any combination thereof.

Aspect 17 pertains to the system of Aspect 3, wherein the crosslinking solution comprises an ionic salt solution.

Aspect 18 pertains to the system of Aspect 17, wherein the ionic salt solution comprises an ionic calcium salt solution.

Aspect 19 pertains to the system of Aspect 18, wherein ethanol is the solvent in the ionic calcium salt solution.

Aspect 20 pertains to the system of Aspect 3, wherein the coating reactor comprises: inputs comprising the emulsification flow channel in communication with the emulsifier, and a coating solution flow channel intersecting the emulsification flow channel; and an output comprising a coated emulsification flow channel in communication with the separator.

Aspect 21 pertains to the system of Aspect 20, wherein the system further comprises a coating solution stock having an input line to the coating reactor.

Aspect 22 pertains to the system of Aspect 21, further comprising a pump disposed between the coating solution stock and the coating reactor.

Aspect 23 pertains to the system of Aspect 21, wherein the coating solution stock input line further comprises a mass flow controller.

Aspect 24 pertains to the system of Aspect 20, wherein the coating solution comprises a polymer coating solution or a peptide coating solution.

Aspect 25 pertains to the system of Aspect 20, wherein the coating reactor is a continuous flow coating reactor.

Aspect 26 pertains to the system of Aspect 20, wherein inputs to the separator comprise: the coated emulsification flow channel in communication with the coating reactor; and a supercritical fluid supply in communication with the coated emulsification flow channel.

Aspect 27 pertains to the system of Aspect 26, wherein outputs from the separator comprise: a solvent evaporation channel, wherein solvents from the coated emulsification are evaporated and removed by the supercritical fluid; and solids comprising cell culture scaffolds.

Aspect 28 pertains to the system of Aspect 26, wherein the supercritical fluid comprises supercritical CO₂.

Aspect 29 pertains to the system of Aspect 26, further comprising a CO₂ stock and pressure regulator.

Aspect 30 pertains to the system of Aspect 29, wherein the CO₂ stock and pressure regulator are external to the housing.

Aspect 31 pertains to the system of Aspect 3, further comprising an alcohol stock and pressure regulator.

Aspect 32 pertains to the system of Aspect 31, wherein the alcohol stock and pressure regulator are external to the housing.

Aspect 33 pertains to the system of Aspect 31, wherein alcohol in the alcohol stock comprises ethanol.

Aspect 34 pertains to the system of Aspect 31, wherein alcohol from the alcohol stock is supplied to a first alcohol wash disposed between the emulsifier and the coating reactor, wherein an emulsification fluid is washed with alcohol after leaving the emulsifier and before entering the coating reactor.

Aspect 35 pertains to the system of Aspect 31, wherein alcohol from the alcohol stock is supplied to a second alcohol wash disposed between the coating reactor and the separator, wherein a coated emulsification fluid is washed with alcohol after leaving the coating reactor and before entering the separator.

Aspect 36 pertains to the system of Aspect 27, wherein the cell culture scaffolds comprise animal-free, digestible cell culture media substrates.

Aspect 37 pertains to the system of Aspect 27, wherein the cell culture scaffolds comprise a polymer bead or slug.

Aspect 38 pertains to the system of Aspect 27, wherein the cell culture scaffolds comprise dissolvable microcarriers.

Aspect 39 pertains to the system of Aspect 38, wherein the dissolvable microcarriers are dissolvable or digestible by an enzyme or chelating agent.

Aspect 40 pertains to the system of Aspect 38, wherein each dissolvable microcarrier comprises a critical dimension of about 300 μm or less.

Aspect 41 pertains to the system of Aspect 1, wherein the system is closed from the atmosphere and aseptic.

Aspect 42 pertains to a method of producing cell culture scaffolds comprising: crosslinking an aqueous organic solution into shaped gels; binding a layer or coating of a cell growth media to the shaped gels; and drying the coated shaped gels to form cell culture scaffolds comprising aerogels functionalized for use as cell growth media.

Aspect 43 pertains to the method of Aspect 42, wherein the aqueous organic solution comprises a polymer solution or a sugar solution.

Aspect 44 pertains to the method of Aspect 42, wherein the aqueous organic solution comprises a polygalacturonic acid (PGA) solution.

Aspect 45 pertains to the method of Aspect 42, wherein the organic solution comprises oils, nonpolar fluids, alcohols, water, surfactants, or any combination thereof.

Aspect 46 pertains to the method of Aspect 42, wherein the method comprises continuous production of cell culture scaffolds.

Aspect 47 pertains to the method of Aspect 42, wherein the cell culture scaffolds are for three-dimensional cell growth applications.

Aspect 48 pertains to the method of Aspect 42, wherein the cell culture scaffolds comprise digestible cell culture scaffolds.

Aspect 49 pertains to the method of Aspect 42, wherein crosslinking, binding, and drying steps are modular processes that occur within a single device.

Aspect 50 pertains to the method of Aspect 42, wherein the crosslinking step comprises introducing the aqueous organic solution to a crosslinking solution through microfluidic channels or pores to form shaped gels in an emulsion, wherein the emulsion comprises the shaped gels as a dispersed phase and a solvent as a continuous phase.

Aspect 51 pertains to the method of Aspect 50, wherein the crosslinking solution comprises a salt solution.

Aspect 52 pertains to the method of Aspect 51, wherein the salt solution comprises a calcium salt solution comprising CaCl₂, CaCO₃, CaSO₄, or a combination thereof in alcohol.

Aspect 53 pertains to the method of Aspect 50, further comprising exposing the shaped gels to an alcohol washing stage.

Aspect 54 pertains to the method of Aspect 42, wherein the binding step comprises binding a cell growth medium to the shaped gel through a cross-linking reaction facilitated by a crosslinking reagent.

Aspect 55 pertains to the method of Aspect 54, wherein the cell growth medium comprises a polymer coating medium or a peptide coating medium.

Aspect 56 pertains to the method of Aspect 54, further comprising exposing the coated shaped gels to an alcohol washing stage.

Aspect 57 pertains to the method of Aspect 50, wherein the drying step further comprises using small pores or membranes in microchannels to separate the coated shaped gels of the dispersed phase from the solvent of the continuous phase of the emulsion.

Aspect 58 pertains to the method of Aspect 57, wherein the coated shaped gels are carried to a separation vessel.

Aspect 59 pertains to the method of Aspect 57, wherein the solvent is removed from the coated shaped gels.

Aspect 60 pertains to the method of Aspect 59, wherein the solvent is removed from the coated shaped gels through supercritical fluid extraction.

Aspect 61 pertains to the method of Aspect 60, further comprising depressurizing and reclaiming the solvent.

It will be appreciated that the various disclosed embodiments may involve particular features, elements, or steps that are described in connection with that particular embodiment. It will also be appreciated that a particular feature, element, or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “an opening” includes examples having two or more such “openings” unless the context clearly indicates otherwise.

All scientific and technical terms used herein have meanings commonly used in the art unless otherwise specified. The definitions provided herein are to facilitate understanding of certain terms used frequently herein and are not meant to limit the scope of the present disclosure.

As used herein, “have,” “having,” “include,” “including,” “comprise,” “comprising,” or the like are used in their open-ended sense, and generally mean “including, but not limited to.”

Ranges may be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a dimension less than 10 mm” and “a dimension less than about 10 mm” both include embodiments of “a dimension less than about 10 mm” as well as “a dimension less than 10 mm.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

Although multiple embodiments of the present disclosure have been described in the Detailed Description, it should be understood that the disclosure is not limited to the disclosed embodiments, but is capable of numerous rearrangements, modifications and substitutions without departing from the disclosure as set forth and defined by the following claims.

1. A system for producing cell culture scaffolds comprising:
a housing; and
a plurality of modular components disposed in a serial arrangement within the housing, the modular components connected through a plurality of microfluidic flow channels,
wherein the modular components comprise an emulsifier, a coating reactor, and a
separator.

2. (canceled)

3. The system of claim 1, wherein the emulsifier comprises:
inputs comprising:
an organic solution flow channel, and
a crosslinking solution flow channel; and
an output comprising an emulsification flow channel in communication with the coating reactor.

4. The system of claim 3, wherein the organic solution flow channel and the crosslinking solution flow channel are in communication through microfluidic pores disposed between the organic solution flow channel and the crosslinking solution flow channel.

5. The system of claim 3, wherein the emulsifier further comprises a porous membrane.

6. The system of claim 5, wherein a critical dimension of each cell culture scaffold is determined by a pore size of the porous membrane.

7. The system of claim 5, wherein the porous membrane is removable and interchangeable.

8. The system of claim 3, wherein the system further comprises:
an organic solution stock having an input line to the emulsifier; and
a crosslinking solution stock having an input line to the emulsifier.

9. The system of claim 8, further comprising a pump disposed between the organic solution stock and the emulsifier, or between the crosslinking solution stock and the emulsifier.

10. The system of claim 9, wherein at least one of the organic solution stock input line and the crosslinking solution stock input line further comprises a mass flow controller.

11-19. (canceled)

20. The system of claim 3, wherein the coating reactor comprises:
inputs comprising
the emulsification flow channel in communication with the emulsifier, and
a coating solution flow channel intersecting the emulsification flow channel; and
an output comprising a coated emulsification flow channel in communication with the separator.

21. The system of claim 20, wherein the system further comprises a coating solution stock having an input line to the coating reactor.

22-24. (canceled)

25. The system of claim 20, wherein the coating reactor is a continuous flow coating reactor.

26. The system of claim 20, wherein inputs to the separator comprise:
the coated emulsification flow channel in communication with the coating reactor; and
a supercritical fluid supply in communication with the coated emulsification flow channel.

27. The system of claim 26, wherein outputs from the separator comprise:
a solvent evaporation channel, wherein solvents from the coated emulsification are evaporated and removed by the supercritical fluid; and
solids comprising cell culture scaffolds.

28-36. (canceled)

37. The system of claim 27, wherein the cell culture scaffolds comprise a polymer bead, a dissolvable microcarrier, or slug.

38-41. (canceled)

42. A method of producing cell culture scaffolds comprising:
crosslinking an aqueous organic solution into shaped gels;
binding a layer or coating of a cell growth media to the shaped gels; and
drying the coated shaped gels to form cell culture scaffolds comprising aerogels functionalized for use as cell growth media.

43. The method of claim 42, wherein the aqueous organic solution comprises a polymer solution or a sugar solution.

44. The method of claim 42, wherein the aqueous organic solution comprises a polygalacturonic acid (PGA) solution.

45. The method of claim 42, wherein the organic solution comprises oils, nonpolar fluids, alcohols, water, surfactants, or any combination thereof.

46. The method of claim 42, wherein the method comprises continuous production of cell culture scaffolds.

47-61. (canceled)
